(19) United States
(12) Patent Application Publication
(10) Pub. No.: US 2022/0266093 A1
(43) Pub. Date: Aug. 25, 2022

(54) Workout Generation System and Method

(71) Applicant: Larkspur, LLC, Boston, MA (US)

(72) Inventor: Julie Lenahan, Boston, MA (US)

(21) Appl. No.: 17/680,898

(22) Filed: Feb. 25, 2022

Related U.S. Application Data
(60) Provisional application No. 63/153,833, filed on Feb. 25, 2021.

(51) Int. Cl. A63B 24/00

(52) U.S. Cl. CPC A63B 24/0075; A63B 2230/015; A63B 2225/50

(57) ABSTRACT

A computer-implemented method, computer program product and computing system for defining one or more physical identifiers for a user; defining one or more physical limitations for the user; defining workout criteria for the user; and generating an online exercise routine for the user based, at least in part, upon the one or more physical identifiers, the one or more physical limitations and the workout criteria.

### RELATED APPLICATION(S)

This application claims the benefit of U.S. Provisional Application No. 63/153,833 filed on 25 Feb. 2021, the entire contents of which are incorporated herein by reference.

### TECHNICAL FIELD

This disclosure relates to workout generation systems and methods and, more particularly, to workout generation systems and methods that consider the physical limitations of the user.

### BACKGROUND

The one hundred billion dollar fitness industry has drastically changed over the last year. Pre-pandemic, about 184 million people had active memberships in in-person gyms, studios, etc. Workouts were conducted in fitness studios and gyms of all sizes across many different types of physical activity: Pilates, cycling, barre, etc. There were a mixture of group classes and private training sessions. Through group classes, the consumer generally had some idea of what the class would entail ahead of participation. For example, if you attend a cycling class, you know you're getting cardio or if you go to yoga, you know you're getting a good stretch. In any of these scenarios, the consumer is responsible for deciding what class they take based on their goals. In addition, within each of these classes, the workout is a one-size-fits-all model. The classes have a format that the consumer must fit into regardless of what their goals may be. Through private training sessions, the instruction can be either one-on-one or a small group. These two formats generally result in a custom workout plan created specifically for an individual's goals but there is a premium price point.

With the start of pandemic-related shutdowns in March of 2020, many fitness professionals, gym owners, and fitness corporations were forced to adapt their delivery method to a virtual environment. This shift to virtual training for clients was crucial to maintaining a business. The shutdowns resulted in thousands of instructors and gyms creating their own online platforms. These platforms hosted a mixture of virtual classes and virtual personal training sessions. These platforms could have hundreds of workouts to choose from. With no shortage of options for fitness consumers, the selection of a particular format and class could be a daunting task. The two major shortcomings of these live social media offerings is that the consumer often selects based on the personality or following of the instructor with no particular knowledge of which muscle groups or difficulty level would be targeted. This format maintains a one-size-fits-all approach. The aforementioned offering lacks customization and do not result in a program tailored specifically to an individual's body and fitness goals. Clearly, the options in the virtual fitness space are plentiful across different types of workout routines and class formats. However and unfortunately, the vast majority of these routines seem to utilize this one-size-fits-all approach that lacks customization.

Specifically, the current virtual fitness offerings ask the user to choose what workout they would like to do during a given day. And with thousands upon thousands of options out there, the choices can be overwhelming and confusing.

### SUMMARY OF DISCLOSURE

In one implementation, a computer-implemented method is executed on a computing device and includes: defining one or more physical identifiers for a user; defining one or more physical limitations for the user; defining workout criteria for the user; and generating an online exercise routine for the user based, at least in part, upon the one or more physical identifiers, the one or more physical limitations and the workout criteria.

One or more of the following features may be included. Generating an online exercise routine for the user based, at least in part, upon the one or more physical identifiers, the one or more physical limitations and the workout criteria may include: combining two of more discrete workout portions to form the online exercise routine. The online exercise routine may be effectuated. Effectuating the online exercise routine may include: streaming the online exercise routine to the user. The one or more physical identifiers for the user may include one or more of: an age of the user; a weight of the user; a height of the user; a gender of the user; and a physical condition of the user. The one or more physical limitations for the user may define one or more of: an area of the body that the user physically cannot exercise; an area of the body that the user chooses not to exercise; an area of the body that the user chooses to lightly exercise; a type of exercise that the user physically cannot perform; and a type of exercise that the user chooses not to perform. The workout criteria may define one or more of: a length of a workout; a target body area; a workout type; and a workout result.

In another implementation, a computer program product resides on a computer readable medium and has a plurality of instructions stored on it. When executed by a processor, the instructions cause the processor to perform operations including defining one or more physical identifiers for a user; defining one or more physical limitations for the user; defining workout criteria for the user; and generating an online exercise routine for the user based, at least in part, upon the one or more physical identifiers, the one or more physical limitations and the workout criteria.

One or more of the following features may be included. Generating an online exercise routine for the user based, at least in part, upon the one or more physical identifiers, the one or more physical limitations and the workout criteria may include: combining two of more discrete workout portions to form the online exercise routine. The online exercise routine may be effectuated. Effectuating the online exercise routine may include: streaming the online exercise routine to the user. The one or more physical identifiers for the user may include one or more of: an age of the user; a weight of the user; a height of the user; a gender of the user; and a physical condition of the user. The one or more physical limitations for the user may define one or more of: an area of the body that the user physically cannot exercise; an area of the body that the user chooses not to exercise; an area of the body that the user chooses to lightly exercise; a type of exercise that the user physically cannot perform; and a type of exercise that the user chooses not to perform. The workout criteria may define one or more of: a length of a workout; a target body area; a workout type; and a workout result.

In another implementation, a computing system includes a processor and a memory system configured to perform operations including defining one or more physical identifiers for a user; defining one or more physical limitations for the user; defining workout criteria for the user; and generating an online exercise routine for the user based, at least in part, upon the one or more physical identifiers, the one or more physical limitations and the workout criteria.

One or more of the following features may be included. Generating an online exercise routine for the user based, at least in part, upon the one or more physical identifiers, the one or more physical limitations and the workout criteria may include: combining two of more discrete workout portions to form the online exercise routine. The online exercise routine may be effectuated. Effectuating the online exercise routine may include: streaming the online exercise routine to the user. The one or more physical identifiers for the user may include one or more of: an age of the user; a weight of the user; a height of the user; a gender of the user; and a physical condition of the user. The one or more physical limitations for the user may define one or more of: an area of the body that the user physically cannot exercise; an area of the body that the user chooses not to exercise; an area of the body that the user chooses to lightly exercise; a type of exercise that the user physically cannot perform; and a type of exercise that the user chooses not to perform. The workout criteria may define one or more of: a length of a workout; a target body area; a workout type; and a workout result.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a distributed computing network including a computing device that executes a workout generation process according to an embodiment of the present disclosure;

FIG. 2 is a diagrammatic view of a user interface rendered by the workout generation process of FIG. 1 according to an embodiment of the present disclosure; and

FIG. 3 is a flowchart of the workout generation process of FIG. 1 according to an embodiment of the present disclosure.

Like reference symbols in the various drawings indicate like elements.

### DETAILED DESCRIPTION OF THE PREFERRED EMBODIMENTS

### System Overview

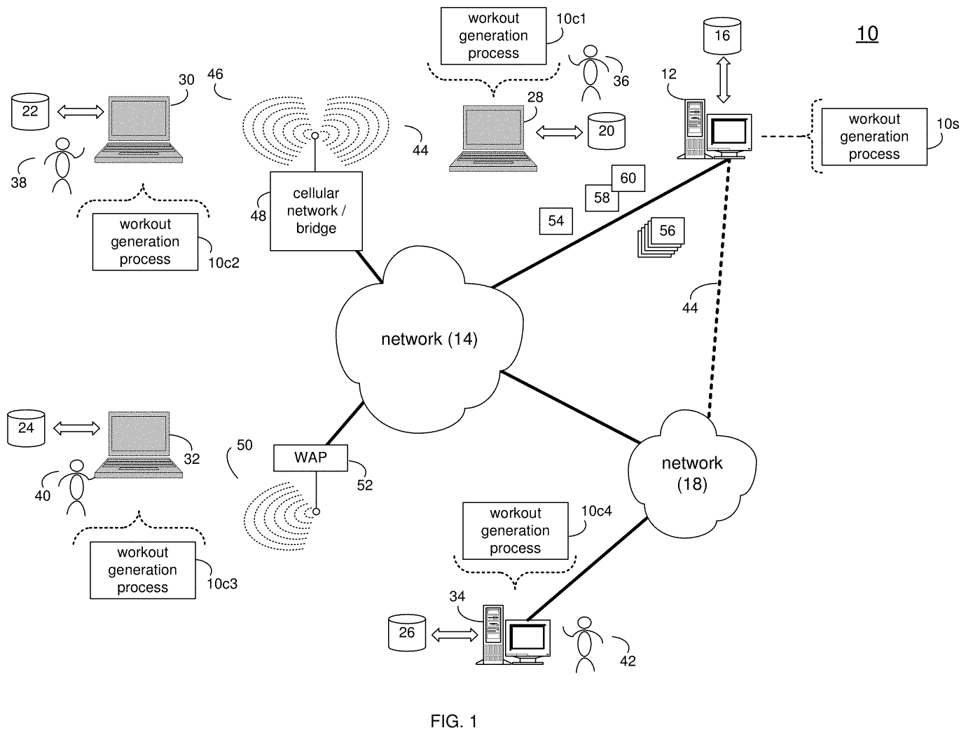

Referring to FIG. 1, there is shown workout generation process 10. Workout generation process 10 may be implemented as a server-side process, a client-side process, or a hybrid server-side/client-side process. For example, workout generation process 10 may be implemented as a purely server-side process via workout generation process 10s. Alternatively, workout generation process 10 may be implemented as a purely client-side process via one or more of workout generation process 10c1, workout generation process 10c2, workout generation process 10c3, and workout generation process 10c4. Alternatively still, workout generation process 10 may be implemented as a hybrid server-side/client-side process via workout generation process 10s in combination with one or more of workout generation process 10c1, workout generation process 10c2, workout generation process 10c3, and workout generation process 10c4. Accordingly, workout generation process 10 as used in this disclosure may include any combination of workout generation process 10s, workout generation process 10c1, workout generation process 10c2, workout generation process 10c3, and workout generation process 10c4.

Workout generation process 10s may be a server application and may reside on and may be executed by computing device 12, which may be connected to network 14 (e.g., the Internet or a local area network). Examples of computing device 12 may include, but are not limited to: a personal computer, a server computer, a series of server computers, a mini computer, a mainframe computer, a smartphone, or a cloud-based computing platform.

The instruction sets and subroutines of workout generation process 10s, which may be stored on storage device 16 coupled to computing device 12, may be executed by one or more processors (not shown) and one or more memory architectures (not shown) included within computing device 12. Examples of storage device 16 may include but are not limited to: a hard disk drive; a RAID device; a random-access memory (RAM); a read-only memory (ROM); and all forms of flash memory storage devices.

Network 14 may be connected to one or more secondary networks (e.g., network 18), examples of which may include but are not limited to: a local area network; a wide area network; or an intranet, for example.

Examples of workout generation processes 10c1, 10c2, 10c3, 10c4 may include but are not limited to a web browser, a game console user interface, a mobile device user interface, or a specialized application (e.g., an application running on e.g., the Android™ platform, the iOS™ platform, the Windows™ platform, the Linux™ platform or the UNIX™ platform). The instruction sets and subroutines of workout generation processes 10c1, 10c2, 10c3, 10c4, which may be stored on storage devices 20, 22, 24, 26 (respectively) coupled to client electronic devices 28, 30, 32, 34 (respectively), may be executed by one or more processors (not shown) and one or more memory architectures (not shown) incorporated into client electronic devices 28, 30, 32, 34 (respectively). Examples of storage devices 20, 22, 24, 26 may include but are not limited to: hard disk drives; RAID devices; random access memories (RAM); read-only memories (ROM), and all forms of flash memory storage devices.

Examples of client electronic devices 28, 30, 32, 34 may include, but are not limited to, a smartphone (not shown), a personal digital assistant (not shown), a tablet computer (not shown), laptop computers 28, 30, 32, personal computer 34, a notebook computer (not shown), a server computer (not shown), a gaming console (not shown), and a dedicated network device (not shown). Client electronic devices 28, 30, 32, 34 may each execute an operating system, examples of which may include but are not limited to Microsoft Windows™, Android™, iOS™, Linux™, or a custom operating system.

Users 36, 38, 40, 42 may access workout generation process 10 directly through network 14 or through secondary network 18. Further, workout generation process 10 may be connected to network 14 through secondary network 18, as illustrated with link line 44.

The various client electronic devices (e.g., client electronic devices 28, 30, 32, 34) may be directly or indirectly coupled to network 14 (or network 18). For example, laptop computer 28 and laptop computer 30 are shown wirelessly coupled to network 14 via wireless communication channels 44, 46 (respectively) established between laptop computers 28, 30 (respectively) and cellular network/bridge 48, which is shown directly coupled to network 14. Further, laptop computer 32 is shown wirelessly coupled to network 14 via wireless communication channel 50 established between laptop computer 32 and wireless access point (i.e., WAP) 52, which is shown directly coupled to network 14. Additionally, personal computer 34 is shown directly coupled to network 18 via a hardwired network connection.

WAP 52 may be, for example, an IEEE 802.11a, 802.11b, 802.11g, 802.11n, Wi-Fi, and/or Bluetooth device that is capable of establishing wireless communication channel 50 between laptop computer 32 and WAP 52. As is known in the art, IEEE 802.11x specifications may use Ethernet protocol and carrier sense multiple access with collision avoidance (i.e., CSMA/CA) for path sharing. As is known in the art, Bluetooth is a telecommunications industry specification that allows e.g., mobile phones, computers, and personal digital assistants to be interconnected using a short-range wireless connection.

### Workout Generation Process

Assume for illustrative purposes that user 36 is a 53-year-old woman who used to exercise regularly but, due to a knee injury, has not exercised in the better part of a year and wishes to get back into the routine of exercising. However, as user 36 travels substantially due to her job, her ability to receive personalized one-on-one training is limited and, therefore, user 36 often resorts to working out in her hotel room during off hours. As will be discussed below in greater detail, user 36 may utilize workout generation process 10 so that a bespoke online exercise routine (e.g., bespoke online exercise routine 54) may be generated for user 36 based upon various specifics (e.g., specifics 56) defined by user 36.

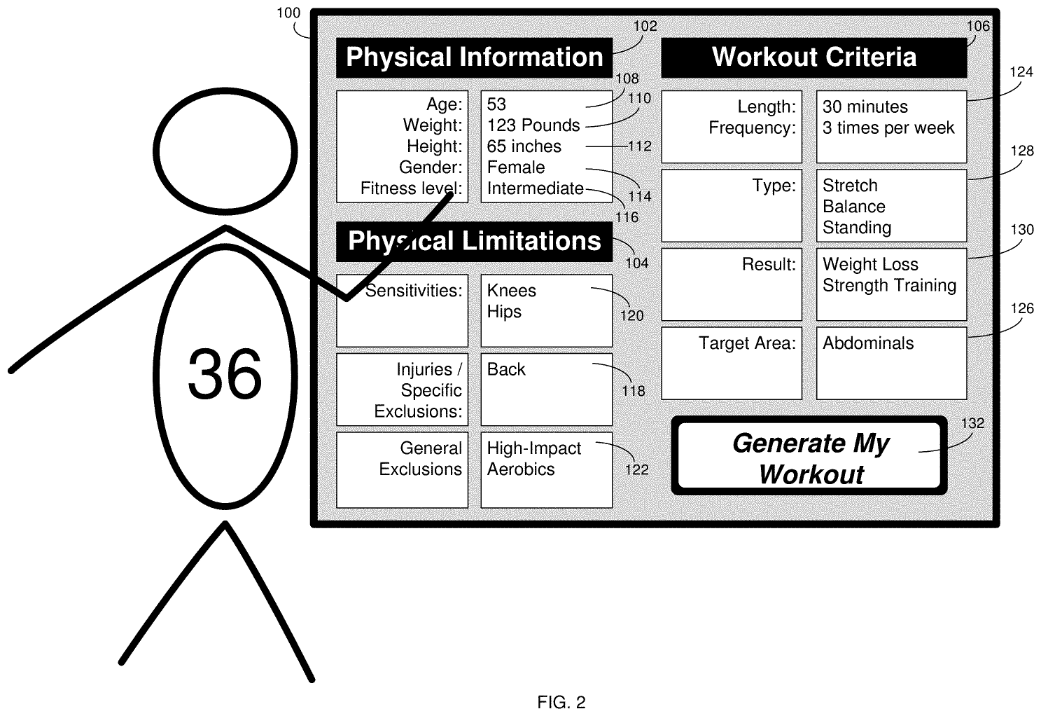

Referring also to FIG. 2, assume that the user (e.g., user 36) may access workout generation process 10 using various modalities, an example of which may include but is not limited to: a website or a specialized application that is accessible via (or executable on) a client electronic device (e.g., laptop computer 28). Once accessed, workout generation process 10 may render user interface 100, wherein user interface 100 may be configured to allow the user (e.g., user 36) to define the various specifics (e.g., specifics 56) that may be used by workout generation process 10 to generate the bespoke online exercise routine (e.g., bespoke online exercise routine 54) for the user (e.g., user 36) as discussed above.

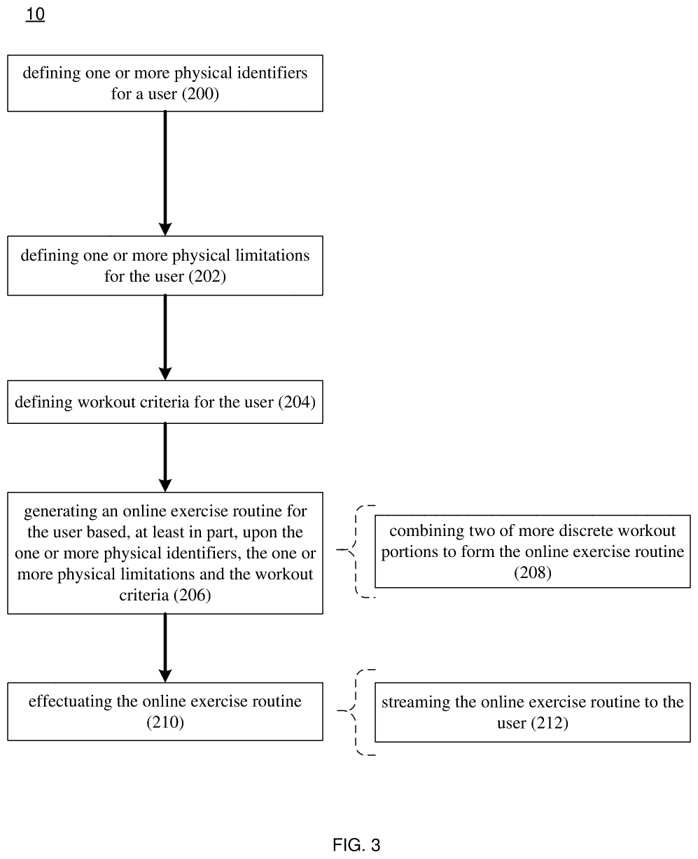

Referring also to FIG. 3, workout generation process 10 may be configured to enable a user (e.g., user 36) to utilize user interface 100 to define the above-referenced various specifics (e.g., specifics 56) that may be used by workout generation process 10 to generate bespoke online exercise routine 54. Specifically, workout generation process 10 may be configured to enable a user (e.g., user 36) to:
define 200 one or more physical identifiers (e.g., physical identifiers 102) for the user (e.g., user 36);
define 202 one or more physical limitations (e.g., physical limitations 104) for the user (e.g., user 36); and/or
define 204 workout criteria (e.g., workout criteria 106) for the user (e.g., user 36);

wherein these physical identifiers (e.g., physical identifiers 102), physical limitations (e.g., physical limitations 104) and/or workout criteria (e.g., workout criteria 106) may be included within specifics 56.

Examples of the one or more physical identifiers (e.g., physical identifiers 102) for the user (e.g., user 36) that may be defined within the above-referenced various specifics (e.g., specifics 56) may include but are not limited to one or more of:
An Age of the User: Through the use of user interface 100 and within field 108, the user (e.g., user 36) may define their age (e.g., in years) so that workout generation process 10 may adjust the level of difficulty of bespoke online exercise routine 54 based upon the age of user 36. In this illustrative example, user 36 is 53 years old.
A Weight of the User: Through the use of user interface 100 and within field 110, the user (e.g., user 36) may define their weight (e.g., in pounds/kilograms/stones) so that workout generation process 10 may adjust the level of difficulty of bespoke online exercise routine 54 based upon the weight of user 36. In this illustrative example, user 36 weighs 123 pounds.
A Height of the User; Through the use of user interface 100 and within field 112, the user (e.g., user 36) may define their height (e.g., in inches/feet/meters/centimeters) so that workout generation process 10 may adjust the level of difficulty of bespoke online exercise routine 54 based upon the height of user 36. In this illustrative example, user 36 is 65 inches tall.
A Gender of the User: Through the use of user interface 100 and within field 114, the user (e.g., user 36) may define their gender (e.g., female/male) so that workout generation process 10 may adjust the level of difficulty of bespoke online exercise routine 54 based upon the gender of user 36. In this illustrative example, user 36 is female.
A Physical Condition of the User: Through the use of user interface 100 and within field 116, the user (e.g., user 36) may define their physical condition (e.g., expert/advanced/intermediate/novice) so that workout generation process 10 may adjust the level of difficulty of bespoke online exercise routine 54 based upon the physical condition of user 36. In this illustrative example, user 36 is an intermediate.

The user (e.g., user 36) may define the above-described physical identifiers (e.g., physical identifiers 102) within user interface 100 using various modalities, examples of which may include but are not limited to: typing, drop down menus, and text-to-speech voice commands.

Examples of the one or more physical limitations (e.g., physical limitations 104) for the user (e.g., user 36) that may be defined within the above-referenced various specifics (e.g., specifics 56) may include but are not limited to one or more of:
An Area that the User Physically Cannot Exercise: Through the use of user interface 100 and within field 118, the user (e.g., user 36) may define areas of their body that they physically cannot exercise (e.g., due to an injury) so that workout generation process 10 may tailor bespoke online exercise routine 54 to exclude such areas. In this illustrative example, user 36 is identifying that they have a back injury. Accordingly, workout generation process 10 may define bespoke online exercise routine 54 so that it excludes exercising the back of user 36.
An Area that the User Chooses not to Exercise: Through the use of user interface 100 and within field 118, the user (e.g., user 36) may define areas of their body that they choose not to exercise so that workout generation process 10 may tailor bespoke online exercise routine 54 to exclude such areas. In this illustrative example, user 36 is identifying that they choose not to exercise their back. Accordingly, workout generation process 10 may define bespoke online exercise routine 54 so that it excludes exercising the back of user 36.
An Area that the User Chooses to Lightly Exercise: Through the use of user interface 100 and within field 120, the user (e.g., user 36) may define areas of their body that they choose to exercise lightly so that workout generation process 10 may tailor bespoke online exercise routine 54 to lightly exercise such areas. In this illustrative example, user 36 is identifying that they have sensitive knees and hips. Accordingly, workout generation process 10 may define bespoke online exercise routine 54 so that it lightly exercises the knees and hips of user 36.
A Type of Exercise that the User Physically Cannot Perform: Through the use of user interface 100 and within field 122, the user (e.g., user 36) may define types of exercise that they physically cannot perform (e.g., due to an injury) so that workout generation process 10 may tailor bespoke online exercise routine 54 to avoid such types of exercise. In this illustrative example, user 36 is identifying that they have sensitive knees and hips. Accordingly, workout generation process 10 may define bespoke online exercise routine 54 so that it excludes high-impact aerobics.
A Type of Exercise that the User Chooses not to Perform: Through the use of user interface 100 and within field 122, the user (e.g., user 36) may define types of exercise that they choose not to perform so that workout generation process 10 may tailor bespoke online exercise routine 54 to avoid such types of exercise. In this illustrative example, user 36 is identifying that they choose not to perform high-impact aerobics. Accordingly, workout generation process 10 may define bespoke online exercise routine 54 so that it excludes high-impact aerobics.

The user (e.g., user 36) may define the above-described physical limitations (e.g., physical limitations 104) within user interface 100 using various modalities, examples of which may include but are not limited to: typing, drop down menus, and text-to-speech voice commands.

Examples of the workout criteria (e.g., workout criteria 106) that may be defined within the above-referenced various specifics (e.g., specifics 56) may include but are not limited to one or more of:
A Length of a Workout: Through the use of user interface 100 and within field 124, the user (e.g., user 36) may define a length for their workout (e.g., in minutes) so that workout generation process 10 may tailor bespoke online exercise routine 54 based upon such a length. In this illustrative example, the length of bespoke online exercise routine 54 is 30 minutes.
A Frequency for the Workout: Through the use of user interface 100 and within field 124, the user (e.g., user 36) may define a frequency for their workout (e.g., once/daily/three times per week/once per week) so that workout generation process 10 may tailor bespoke online exercise routine 54 based upon such a frequency. In this illustrative example, the frequency of bespoke online exercise routine 54 is three times per week.
A Target Body Area: Through the use of user interface 100 and within field 126, the user (e.g., user 36) may define a target body area for their workout (e.g., arms/legs/abdominals/glutes) so that workout generation process 10 may tailor bespoke online exercise routine 54 based upon such a target body area. In this illustrative example, the target body area of bespoke online exercise routine 54 is the abdominals of user 36
A Workout Type: Through the use of user interface 100 and within field 128, the user (e.g., user 36) may define a type for their workout (e.g., stretch/balance/standing/endurance/cardio) so that workout generation process 10 may tailor bespoke online exercise routine 54 based upon such a workout type. In this illustrative example, the workout type of bespoke online exercise routine 54 is stretch/balance/standing.
A Workout Result: Through the use of user interface 100 and within field 130, the user (e.g., user 36) may define a desired result for their workout (e.g., weight loss / strength training / flexibility) so that workout generation process 10 may tailor bespoke online exercise routine 54 based upon such a desired result. In this illustrative example, the desired result of bespoke online exercise routine 54 is weight loss/strength training.

The user (e.g., user 36) may define the above-described workout criteria (e.g., workout criteria 106) within user interface 100 using various modalities, examples of which may include but are not limited to: typing, drop down menus, and text-to-speech voice commands.

Once these physical identifiers (e.g., physical identifiers 102), physical limitations (e.g., physical limitations 104) and/or workout criteria (e.g., workout criteria 106) are defined, workout generation process 10 may generate 206 the online exercise routine (e.g., bespoke online exercise routine 54) for the user (e.g., user 36) based, at least in part, upon the physical identifiers (e.g., physical identifiers 102), the physical limitations (e.g., physical limitations 104) and/or the workout criteria (e.g., workout criteria 106). For example and once physical identifiers 102, physical limitations 104 and/or workout criteria 106 are defined, user 36 may select “Generate my Workout” button 132 within user interface 100 to generate 206 the online exercise routine (e.g., bespoke online exercise routine 54) for the user (e.g., user 36).

When generating 206 the online exercise routine (e.g., bespoke online exercise routine 54) for the user (e.g., user 36) based, at least in part, upon the physical identifiers (e.g., physical identifiers 102), the physical limitations (e.g., physical limitations 104) and/or the workout criteria (e.g., workout criteria 106), workout generation process 10 may combine 208 two of more discrete workout portions (e.g., discrete workout portions 58, 60) to form the online exercise routine (e.g., bespoke online exercise routine 54). As discussed above and in this illustrative example, the workout type of bespoke online exercise routine 54 was defined by user 36 to be stretch & balance training while standing. Accordingly, workout generation process 10 may combine 208 a first 15-minute discrete workout portion (e.g., discrete workout portion 58) that concerns stretch training (while standing) and a second 15-minute discrete workout portion (e.g., discrete workout portion 60) that concerns balance training (while standing) to form a 30-minute online exercise routine (e.g., bespoke online exercise routine 54) that concerns stretch & balance training while standing.

Once generated 206, workout generation process 10 may effectuate 210 the online exercise routine (e.g., bespoke online exercise routine 54). For example and when effectuating the online exercise routine (e.g., bespoke online exercise routine 54), workout generation process 10 may stream 212 the online exercise routine (e.g., bespoke online exercise routine 54) to the user (e.g., user 36) and/or allow the user (e.g., user 36) to download the online exercise routine (e.g., bespoke online exercise routine 54) to (in this example) laptop computer 28.

### General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
defining one or more physical identifiers for a user;
defining one or more physical limitations for the user;
defining workout criteria for the user; and
generating an online exercise routine for the user based, at least in part, upon the one or more physical identifiers, the one or more physical limitations and the workout criteria.

2. The computer-implemented method of claim 1 wherein generating an online exercise routine for the user based, at least in part, upon the one or more physical identifiers, the one or more physical limitations and the workout criteria includes:
combining two of more discrete workout portions to form the online exercise routine.

3. The computer-implemented method of claim 1 further comprising:
effectuating the online exercise routine.

4. The computer-implemented method of claim 3 wherein effectuating the online exercise routine includes:
streaming the online exercise routine to the user.

5. The computer-implemented method of claim 1 wherein the one or more physical identifiers for the user include one or more of:
an age of the user;
a weight of the user;
a height of the user;
a gender of the user; and
a physical condition of the user.

6. The computer-implemented method of claim 1 wherein the one or more physical limitations for the user define one or more of:
an area of the body that the user physically cannot exercise;
an area of the body that the user chooses not to exercise;
an area of the body that the user chooses to lightly exercise;
a type of exercise that the user physically cannot perform; and
a type of exercise that the user chooses not to perform.

7. The computer-implemented method of claim 1 wherein the workout criteria define one or more of:
a length of a workout;
a target body area;
a workout type; and
a workout result.

8. A computer program product residing on a computer readable medium having a plurality of instructions stored thereon which, when executed by a processor, cause the processor to perform operations comprising:
defining one or more physical identifiers for a user;
defining one or more physical limitations for the user;
defining workout criteria for the user; and
generating an online exercise routine for the user based, at least in part, upon the one or more physical identifiers, the one or more physical limitations and the workout criteria.

9. The computer-implemented method of claim 8 wherein generating an online exercise routine for the user based, at least in part, upon the one or more physical identifiers, the one or more physical limitations and the workout criteria includes:
combining two of more discrete workout portions to form the online exercise routine.

10. The computer-implemented method of claim 8 further comprising:
effectuating the online exercise routine.

11. The computer-implemented method of claim 10 wherein effectuating the online exercise routine includes:
streaming the online exercise routine to the user.

12. The computer-implemented method of claim 8 wherein the one or more physical identifiers for the user include one or more of:
an age of the user;
a weight of the user;
a height of the user;
a gender of the user; and
a physical condition of the user.

13. The computer-implemented method of claim 8 wherein the one or more physical limitations for the user define one or more of:
an area of the body that the user physically cannot exercise;
an area of the body that the user chooses not to exercise;
an area of the body that the user chooses to lightly exercise;
a type of exercise that the user physically cannot perform; and
a type of exercise that the user chooses not to perform.

14. The computer-implemented method of claim 8 wherein the workout criteria define one or more of:
a length of a workout;
a target body area;
a workout type; and
a workout result.

15. A computing system including a processor and memory configured to perform operations comprising:
defining one or more physical identifiers for a user;
defining one or more physical limitations for the user;
defining workout criteria for the user; and
generating an online exercise routine for the user based, at least in part, upon the one or more physical identifiers, the one or more physical limitations and the workout criteria.

16. The computer-implemented method of claim 15 wherein generating an online exercise routine for the user based, at least in part, upon the one or more physical identifiers, the one or more physical limitations and the workout criteria includes:
combining two of more discrete workout portions to form the online exercise routine.

17. The computer-implemented method of claim 15 further comprising:
effectuating the online exercise routine.

18. The computer-implemented method of claim 17 wherein effectuating the online exercise routine includes:
streaming the online exercise routine to the user.

19. The computer-implemented method of claim 15 wherein the one or more physical identifiers for the user include one or more of:
an age of the user;
a weight of the user;
a height of the user;
a gender of the user; and
a physical condition of the user.

20. The computer-implemented method of claim 15 wherein the one or more physical limitations for the user define one or more of:
an area of the body that the user physically cannot exercise;
an area of the body that the user chooses not to exercise;
an area of the body that the user chooses to lightly exercise;
a type of exercise that the user physically cannot perform; and
a type of exercise that the user chooses not to perform.

21. The computer-implemented method of claim 15 wherein the workout criteria define one or more of:
a length of a workout;
a target body area;
a workout type; and
a workout result.
